(19) United States
(12) Patent Application Publication
(10) Pub. No.: US 2018/0353592 A1
(43) Pub. Date: Dec. 13, 2018

(54) Combined application of Haemophilus parasuis LC strain and Haemophilus parasuis LZ-20100109 stain

(71) Applicant: INSTITUTE OF ANIMAL SCIENECE AND VETERINARY MEDICINE SHANDONG ACADEMY OF AGRICULTARAL SCIENCES, Jinan, Shandong (CN)

(72) Inventors: Jiaqiang Wu, Jinan, Shadong (CN); Yuyu Zhang, Jinan, Shadong (CN); Jiang Yu, Jinan, Shadong (CN); Ke Wang, Jinan, Shadong (CN); Yijun Du, Jinan, Shadong (CN); Wenbo Sun, Jinan, Shadong (CN); Jun Li, Jinan, Shadong (CN); Jinban Wang, Jinan, Shadong (CN)

(21) Appl. No.: 14/917,011

(22) PCT Filed: Jan. 29, 2015
(86) PCT No.: PCT/CN2015/000058

§ 371 Date: Mar. 6, 2016

(51) Int. Cl. A61K 39/116; A61K 39/39; C12N 1/20; A61K 39/102; A61K 39/02

(52) U.S. Cl. CPC A61K 39/116; A61K 39/39; A61P 31/04; A61K 39/102; A61K 39/0208; C12N 1/20

(57) ABSTRACT

A combined application of Haemophilus parasuis (HPS) LC strain having a deposit number of CGMCC No. 5257 and HPS LZ-20100109 strain having a deposit number of CGMCC No. 5802 in preparing a bivalent inactivated vaccine is provided, relating to HPS disease vaccines in a field of veterinary biologics. The combined application of the HPS LC strain and the HPS LZ-20100109 strain in preparing the bivalent inactivated vaccine is safe and reliable, providing not only a homologous challenge protection against serotype 1 and serotype 5, but also certain cross protection against heterologous challenges of serotype 2, serotype 4, serotype 10, serotype 12, serotype 13, serotype 14, and serotype 15 of HPS. The combined application of the HPS LC strain and the HPS LZ-20100109 strain has an obviously increased effect. After immunizing swine, a relatively strong immunity is generated; an incidence rate and a mortality rate of inoculated swine decrease obviously.

### CROSS REFERENCE OF RELATED APPLICATION

This is a U.S. National Stage under 35 U.S.C 371 of the International Application PCT/CN2015/000058, filed Jan. 29, 2015.

### BACKGROUND OF THE PRESENT INVENTION

### Field of Invention

The present invention relates to Haemophilus parasuis (HPS) disease vaccines in a field of veterinary biologics, and more particularly to a combined application of HPS LC strain and HPS LZ-20100109 strain in preparing a bivalent inactivated vaccine.

### Description of Related Arts

The HPS is a kind of the gram-negative small bacillus, which is nicotinamide-adenine-dinucleotide-dependent (NAD-dependent) and nonmotile and belongs to haemophilus of pasteurellaceae. The HPS often causes polyserositis, arthritis and meningitis on the swine, and the disease caused by the HPS is also called Glasser's disease. According to the survey, the HPS can affect the young swine, aged between 2 weeks and 4 months; the HPS disease mainly occurs before and after weaning and during the nursing period, and is commonly seen on the swine aged between 5 weeks and 8 weeks; the incidence rate reaches 40%, and, in severe cases, the mortality rate reaches 50%. The HPS can also infect the parturient sow, causing abortion, stillborn fetus and weak piglets. With the continuous development of the swine industry, the scale and the intensity of the swine farm increase continuously; the stocking density of the swine becomes higher and higher; and the HPS infection increases obviously, which has become the major cause of the disease incidence and the death of the weaned piglets in the swine farm. Moreover, some immunosuppressive diseases, such as the porcine reproductive and respiratory syndrome and the porcine circovirus disease, often infect the swine with the HPS or cause the secondary infection, causing a huge economic loss of the global swine industry, and bringing about a greater difficulty in the diagnosis and the comprehensive prevention and treatment of the HPS disease.

In recent years, the HPS disease has become the major threat to the swine industry in China. The drug abuse causes the increasingly widespread drug resistance of the bacteria. The vaccine immunization is an effective way for preventing and controlling the HPS disease. The serotypes of the HPS are numerous; the virulence and the pathogenicity of different serotype strains have a relatively large difference; the immune cross protection among each serotype is low; and the strains separated in different areas have relatively strong local characteristics. Thus, the single-strain vaccine has the limited resistance against the HPS disease. Moreover, the conventional commercial HPS vaccines all over the world are unable to provide a completely effective immune protection against the HPS disease. In order to prevent and control the HPS disease, it is urgent to provide more HPS strains having the good cross protection and the good immunogenicity and to prepare the highly-efficient bivalent or multivalent HPS vaccine with the HPS strains.

The Chinese patent publication, CN102399724A, disclosed the HPS LC strain. The HPS LC strain has a relatively strong pathogenicity to the swine and a good immunogenicity. The inactivated aluminum hydroxide gel vaccine prepared with the HPS LC strain is safe and reliable. The inactivated aluminum hydroxide gel vaccine provides not only the homologous challenge protection but also certain cross protection against the heterologous challenges of serotype 4, serotype 5, serotype 10, serotype 12, serotype 14, and serotype 15 of the HPS. After immunization, a relatively strong immunity is generated; the incidence rate and the mortality rate of the inoculated swine decrease obviously.

The Chinese patent publication, CN102851249A, disclosed the HPS LZ-20100109 strain and applications thereof. The HPS LZ-20100109 strain has a relatively strong pathogenicity to the swine and a good immunogenicity. The inactivated aluminum hydroxide gel vaccine prepared with the HPS LZ-20100109 strain is safe and reliable. The inactivated aluminum hydroxide gel vaccine provides not only the homologous challenge protection but also certain cross protection against the heterologous challenges of serotype 1, serotype 2, serotype 4, serotype 12, serotype 14, and serotype 15 of the HPS. After immunization, a relatively strong immunity is generated; the incidence rate and the mortality rate of the inoculated swine decrease obviously.

However, the above two disclosed vaccines fail to exhibit good cross protection rate for all the pathogenic serotypes of the HPS. For example, both of the two vaccines have a low protection rate for the serotype 13 of the HPS. Thus, to provide a vaccine which has a good cross protection rate for all HPS pathogenic serotypes is necessary and has a bright application prospect.

### SUMMARY OF THE PRESENT INVENTION

An object of the present invention is to provide a combined application of HPS LC strain and HPS LZ-20100109 strain in preparing a bivalent inactivated vaccine which has a strong immune cross protection among each serotype, so as to overcome the low immune cross protection among each serotype and the weak immunogenicity of the conventional commercial HPS vaccines all over the world.

Technical solutions of the present invention are described as follows.

An HPS LC strain which is a serotype 1 strain has a deposit number of CGMCC No. 5257.

An HPS LZ-20100109 strain which is a serotype 5 strain has a deposit number of CGMCC No. 5802.

A combined application of the HPS LC strain and the HPS LZ-20100109 strain in preparing a bivalent inactivated vaccine of HPS disease comprises steps of: choosing the HPS LC strain and the EPS LZ-20100109 strain; after activating, respectively inoculating the HPS LC strain and the HPS LZ-20100109 strain into suitable media for culturing; obtaining cultures; inactivating the cultures with formaldehyde solution; and adding aluminum hydroxide gel into the cultures to obtain the bivalent inactivated vaccine of the HPS disease.

Preferably, the bivalent inactivated vaccine of the HPS disease is an aluminum hydroxide gel vaccine.

The HPS LC strain and the HPS LZ-20100109 strain in the aluminum hydroxide gel vaccine have a total antigen content≥2.5×10⁹ CFU/mL.

A ratio of an antigen content of the HPS LC strain to an antigen content of the HPS LZ-20100109 strain in the aluminum hydroxide gel vaccine is (2:3)-(3:2).

Preferably, the ratio of the antigen content of the HPS LC strain to the antigen content of the HPS LZ-20100109 strain in the aluminum hydroxide gel vaccine is 2:3, 1:1, or 3:2.

The present invention has following advantages. The bivalent inactivated vaccine prepared through the combined application of the HPS LC strain and the HPS LZ-20100109 strain is safe and reliable. The bivalent inactivated vaccine provides not only the homologous challenge protection against serotype 1 and serotype 5, but also the relatively good cross protection against the heterologous challenges of serotype 2, serotype 4, serotype 10, serotype 12, serotype 13, serotype 14, and serotype 15 of the HPS. The combined application of the HPS LC strain and the HPS LZ-20100109 strain has an obviously increased effect. The bivalent inactivated vaccine (serotype 1 LC strain+serotype 5 LZ-20100109 strain) of the HPS disease generates a relatively strong immunity after immunizing the swine. The incidence rate and the mortality rate of the inoculated swine decrease obviously. An immune effect of the bivalent inactivated vaccine prepared by the present invention is equal to or better than the immune effect of the conventional commercial vaccines on the market. The bivalent inactivated vaccine has advantages in competition with similar products all over the world. The bivalent inactivated vaccine is able to effectively prevent prevalence and spread of the HPS disease and decrease an economic loss caused by the HPS disease, showing a wide application prospect.

These and other objectives, features, and advantages of the present invention will become apparent from the following detailed description, the accompanying drawings, and the appended claims.

### DETAILED DESCRIPTION OF THE PREFERRED EMBODIMENT

The present invention is further illustrated with the following examples.

HPS LC strain, serotype 1, has a deposit number of CGMCC No. 5257, deposited in China General Microbiological Culture Collection Center (CGMCC).

HPS LZ-20100109 strain, serotype 5, has a deposit number of CGMCC No. 5802, deposited in the CGMCC.

### Example 1

### Media for Production

Preparing tryptone soy agar (TSA) medium: weighing 4 g TSA powder and adding the 4 g TSA powder into 100 mL distilled water; after fully mixing, sterilizing at a temperature of 121° C. for 15 min; cooling to 50° C.-60° C., and thereafter adding nicotinamide adenine dinucleotide (NAD) having a final concentration of 0.001% and new-born calf serum (NBCS) having a final concentration of 5%; and, after uniformly mixing, pouring to form a plate.

Preparing tryptone soy broth (TSB) medium: weighing 3 g TSB powder and adding the 3 g TSB powder into 100 mL distilled water; sterilizing at the temperature of 121° C. for 15 min. The NAD having the final concentration of 0.001% and the NBCS having the final concentration of 5% were added into the TSB medium and mixed uniformly, instantly before use.

A combined application of the HPS LC strain and the HPS LZ-20100109 strain in preparing a bivalent inactivated vaccine of EPS disease comprises steps of: choosing the HPS LC strain, deposit number CGMCC No. 5257, and the HPS LZ-20100109 strain, deposit number CGMCC No. 5802; after activating, respectively inoculating the HPS LC to strain and the HPS LZ-20100109 strain into suitable media for culturing; obtaining cultures; inactivating the cultures with formaldehyde solution; and adding aluminum hydroxide gel into the cultures for obtaining the bivalent inactivated vaccine of the HPS disease.

The combined application of the HPS LC strain and the HPS LZ-20100109 strain in preparing the bivalent inactivated vaccine of the HPS disease is preferably embodied to comprise the following steps.

1.1 Preparing Seeds for Production

1.1.1 Propagating First-Grade Seeds

Choosing the HPS LC strain, deposit number CGMCC No. 5257, and the HPS LZ-20100109 strain, deposit number CGMCC No. 5802; respectively streak-inoculating the HPS LC strain and the HPS LZ-20100109 strain into first TSA media, and culturing at a temperature of 37° C. for 24-36 h; respectively intensively streak-inoculating single colonies of activated HPS LC strain and activated HPS LZ-20100109 strain into second TSA media, and culturing at the temperature of 37° C. for 16-20 h; washing the second TSA media with phosphate buffered saline (PBS); collecting first bacterial liquids of the HPS LC strain and the HPS LZ-20100109 strain; adding glycerol having a final concentration of 20% into the first bacterial liquids; and, after passing a purity test, preserving below −20° C. for no more than 1 month to serve as the first-grade seeds of the HPS LC strain and the HPS LZ-20100109 strain.

1.1.2 Propagating Second-Grade Seeds

Respectively inoculating the first-grade seeds of the HPS LC strain and the HPS LZ-20100109 strain into TSB media, wherein the inoculated first-grade seeds were 1% of a total volume of the TSB media; shake-culturing the first-grade seeds of the HPS LC strain and the HPS LZ-20100109 strain at the temperature of 37° C. with a rotation speed of 180 r/min, wherein the first-grade seeds of the HPS LC strain were cultured for 12-14 h and the first-grade seeds of the EPS LZ-20100109 strain were cultured for 16-18 h; and, after passing the purity test, namely no other bacteria being detected by microscopy, obtaining the second-grade seeds of the HPS LC strain and the HPS LZ-20100109 strain, which were preserved at a temperature of 2-8° C. for no more than 8 h.

1.2 Preparing Second Bacterial Liquids for Preparing the Bivalent Inactivated Vaccine of the HPS Disease

Aerobically culturing the second-grade seeds of the HPS LC strain and the HPS LZ-20100109 strain with fermentation tanks; respectively inoculating the second-grade seeds of the HPS LC strain and the HPS LZ-20100109 strain, which were liquids, into TSB media, wherein the inoculated second-grade seeds were 1% of a total volume of the TSB media; adding an appropriate amount of defoamer into the TSB media; gradually increasing an aeration volume; culturing the second-grade seeds of the HPS LC strain at the temperature of 37° C. for 12 h, and culturing the second-grade seeds of the HPS LZ-20100109 strain at the temperature of 37° C. for 14 h, so as to obtain the second bacterial liquids of the HPS LZ strain and the HPS LZ-20100109 strain. After finishing culturing the second bacterial liquids, sampling for the purity test and testing according to appendixes of existing Veterinary Pharmacopoeia of the People's Republic of China, wherein a test result was pure; and meanwhile, counting viable bacteria according to the appendixes of the existing Veterinary Pharmacopoeia of the People's Republic of China respectively with TSA media to serve as references for preparing the bivalent inactivated vaccine of the HPS disease.

1.3 Inactivating the Second Bacterial Liquids

Adding the formaldehyde solution having a final concentration of 0.3% into the second bacterial liquids according to a volume ratio, and inactivating at the temperature of 37° C. for 24 h, so as to obtain inactivated bacterial liquids of the HPS LC strain and the HPS LZ-20100109 strain, wherein the inactivated bacterial liquids of the HPS LC strain and the HPS LZ-20100109 strain were respectively inoculated into TSA media for an inactivation test and a result of the inactivation test is required to be sterility.

1.4 Preparing the Bivalent Inactivated Vaccine of the HPS Disease

Mixing the inactivated bacterial liquids of the HPS LC strain with the HPS LZ-20100109 strain in an appropriate proportion to obtain a mixed bacterial liquid; preparing the bivalent inactivated vaccine of the HPS disease according to a ratio, 2:1, of the mixed bacterial liquid to sterilized 20% aluminum hydroxide gel which was added, and meanwhile adding merthiolate which was 0.005% of a total volume of the bivalent inactivated vaccine into the bivalent inactivated vaccine; after fully stirring and mixing, standing at the temperature of 2-8° C. for 1-2 days; and removing supernatant by extracting, so as to condense the bivalent inactivated vaccine into about one-third of the total volume. The bivalent inactivated vaccine had a total antigen content of 2.5×10⁹ CFU/mL.

1.5 Sub-Packaging

Quantitatively sub-packaging and sealing with covers, wherein: during sub-packaging, stirring at any time was necessary, for uniformly mixing the bivalent inactivated vaccine.

1.6 Testing Vaccine Products

1.6.1 Measuring Physical Properties

After standing, the vaccine product showed an upper layer which was a transparent clear liquid and a lower layer which was grayish white precipitation. By fully shaking, the vaccine product became a uniformly mixed suspension liquid.

1.6.2 Sterility Test

The bivalent inactivated vaccine was tested according to the appendixes of the existing Veterinary Pharmacopoeia of the People's Republic of China, and a test result was sterility.

1.6.3 Testing Residual Volumes of Formaldehyde and Mercury-Containing Preservative

The bivalent inactivated vaccine was tested according to the appendixes of the existing Veterinary Pharmacopoeia of the People's Republic of China, and passed the test.

1.6.4 Safety Test

Choosing 5 healthy susceptible piglets, aged between 3-4 weeks; respectively intramuscularly injecting 4 mL bivalent inactivated vaccine into neck of each piglet; observing the piglets for 2 weeks, and obtaining observation results that: the inoculated piglets all had a normal temperature, spirit and appetite, and were healthy; and no adverse reaction caused by the vaccine happened at injection sites.

1.6.5 Efficacy Test

Choosing 20 healthy susceptible piglets, aged between 3-4 weeks, and dividing into 4 groups, wherein each group had 5 piglets; intramuscularly injecting 2.0 mL bivalent inactivated vaccine into neck of each piglet of two groups which served as an immune group, and intramuscularly injecting 2.0 mL PBS into neck of each piglet of the other two groups which served as a control group for comparison; after 21 days, injecting each piglet again with the same dose in the same manner; after 2 weeks, collecting blood of each piglet, separating serum from the blood of each piglet, and measuring a titer of a serum antibody through a micro agglutination test, wherein geometric mean titers of serotype 1 antibodies and serotype 5 antibodies of the piglets of the immune group≥1:32, and geometric mean titers of serotype 1 antibodies and serotype 5 antibodies of the piglets of the control group≤1:4. After collecting the blood of each piglet, respectively choosing 5 piglets from the immune group and the control group, and intraperitoneally injecting 2.0 mL the second bacterial liquid of the HPS LC strain (about 4.5×10⁹ CFU) into each chosen piglet; continuously observing for 14 days, wherein: during the 14 days, dying piglets of the ten chosen piglets were processed with autopsy; disease incidence or death occurred on at least four chosen piglets of the control group; and at least four chosen piglets of the immune group were protected from the HPS disease. Respectively choosing 5 piglets from the immune group and the control group again, and intraperitoneally injecting 2.0 mL the second bacterial liquid of the HPS LZ-20100109 strain (about 4.5×10⁹ CFU) into each chosen piglet; continuously observing for 14 days, wherein: during the 14 days, dying piglets of the ten chosen piglets were processed with the autopsy; the disease incidence or the death occurred on at least four chosen piglets of the control group; and at least four chosen piglets of the immune group were protected from the HPS disease.

### Example 2

### Efficacy Comparison Test Between Bivalent Inactivated Vaccine and Univalent Inactivated Vaccine of HPS Disease

2.1 Preparation of Bivalent Inactivated Vaccine

The bivalent inactivated vaccine was prepared as illustrated in the example 1. The inactivated bacterial liquids of the HPS LC strain and the HPS LZ-20100109 strain were mixed in the appropriate proportion. The ratios of the antigen content of the HPS LC strain to the antigen content of the HPS LZ-20100109 strain in the bivalent inactivated vaccines were respectively 2:3, 1:1, and 3:2, but the total antigen content of the HPS LC strain and the HPS LZ-20100109 strain was 2.5×10⁹ CFU/mL. The bivalent inactivated vaccines of the three ratios were respectively called bivalent inactivated vaccine I, bivalent inactivated vaccine II, and bivalent inactivated vaccine III.

2.2 Preparation of Univalent Inactivated Vaccine

The univalent inactivated vaccine was prepared as illustrated in the example 1, except that bacteria contents of the inactivated bacterial liquids of the EPS LC strain and the HPS LZ-20100109 strain were firstly adjusted to about 2.5×10⁹ CFU/mL, and thereafter the univalent inactivated vaccine was prepared with the ratio of the inactivated bacterial liquid to the sterilized 20% aluminum hydroxide gel being 2:1. A univalent inactivated vaccine product had an antigen content of 2.5×10⁹ CFU/mL.

2.3 Homologous Protection Test

The homologous protection test for each serotype included steps of: randomly choosing 30 healthy susceptible piglets, aged between 3-4 weeks, and randomly dividing into 6 groups, wherein each group had 5 piglets; intramuscularly injecting 2.0 mL bivalent inactivated vaccine I into neck of each piglet in one group, intramuscularly injecting 2.0 mL bivalent inactivated vaccine II into neck of each piglet in one group, intramuscularly injecting 2.0 mL bivalent inactivated vaccine III into neck of each piglet in one group, intramuscularly injecting 2.0 mL first univalent inactivated vaccine (serotype 1 LC strain) into neck of each piglet in one group which was a first univalent inactivated vaccine immune group, intramuscularly injecting 2.0 mL second univalent inactivated vaccine (serotype 5 LZ-20100109 strain) into neck of each piglet in one group which was a second univalent inactivated vaccine immune group, and intramuscularly injecting 2.0 mL PBS into neck of each piglet in one group which was a control group; after 3 weeks, injecting piglets of each group again with the same dose in the same manner for strengthening immunity. After 2 weeks, collecting blood of each piglet, separating serum from the blood of each piglet, and measuring a titer of a serum antibody through a micro agglutination test. After collecting the blood of each piglet, intraperitoneally injecting the second bacterial liquid of the HPS LC strain or the HPS LZ-20100109 (about 4.5×10⁹ CFU) into each piglet; after challenge, continuously observing the piglets for 2 weeks and recording disease incidence and death of the piglets. After the challenge, the disease incidence of the piglets was determined upon the following clinical symptoms and lesions in autopsy. The clinical symptoms, such as a temperature increase of more than 0.5° C. for continuous 2 days, depression, irregular hair, and dyspnea; for worse, arthrocele, claudication, dysstasia, neurological symptoms or death. The lesions in the autopsy, such as pericardial effusion, synovial fluid increase and even gelatinous effusion in an articular cavity of a hind leg; for worse, pericardium thickening, fibrinous effusion on a surface of heart and lungs or in abdomen.

Protection effects of the bivalent inactivated vaccines and the univalent inactivated vaccines of the HPS disease against the serotype 1 are showed in Table 1. The disease incidence occurs on 100% (5/5) of the piglets of the control group; all serotype 1 antibodies of the piglets injected with the three bivalent inactivated vaccines, namely the piglets of the bivalent inactivate vaccine immune groups, have a geometric mean titer≥1:32 and a protection rate above 80% (4/5); all serotype 1 antibodies of the piglets of the first univalent inactivated vaccine (serotype 1 LC strain) immune group have a geometric mean titer≥1:32 and a protection rate above 80% (4/5); serotype 1 antibodies of the piglets of the second univalent inactivated vaccine (serotype 5 LZ-20100109 strain) immune group merely have a geometric mean titer of 1:16 and a protection rate of 60% (3/5).

TABLE 1
Protection effects of bivalent inactivated vaccines and univalent inactivated vaccines
of BPS disease against serotype 1

| | | | | | Second | |
|---|---|---|---|---|---|---|
| | | | | First | univalent | |
| | | | | univalent | inactivated | |
| | | | | inactivated | vaccine | |
| | Bivalent | Bivalent | Bivalent | vaccine | (serotype 5 | PBS |
| | inactivated | inactivated | inactivated | (serotype | LZ-20100109 | control |
| Test group | vaccine I | vaccine II | vaccine III | 1 LC strain) | strain) | group |
| Number | 5 | 5 | 5 | 5 | 5 | 5 |
| of piglets | | | | | | |
| Challenge | LC strain | | | | | |
| strain | | | | | | |
| Serotype 1 | 1:32 | 1:37 | 1:42 | 1:37 | 1:16 | |
| antibody | | | | | | |
| titer | | | | | | |
| Number | 1 | 0 | 0 | 0 | 2 | 5 |
| of sick | | | | | | |
| piglets | | | | | | |
| Number | 0 | 0 | 0 | 0 | 0 | 1 |
| of dead | | | | | | |
| piglets | | | | | | |
| Number | 5 | 5 | 5 | 5 | 3 | 0 |
| of healthy | | | | | | |
| piglets | | | | | | |
| Result | 4/5 | 5/5 | 5/5 | 5/5 | 3/5 | 5/5 |
| | Protection | Protection | Protection | Protection | Protection | Incidence |

Protection effects of the bivalent inactivated vaccines and the univalent inactivated vaccines of the HPS disease against the serotype 5 are showed in Table 2. The disease incidence occurs 100% (5/5) of the piglets of the control group; all serotype 5 antibodies of the piglets of the bivalent inactivate vaccine immune group have a geometric mean titer≥1:32 and a protection rate above 80% (4/5); all serotype 5 antibodies of the piglets of the second univalent inactivated vaccine (serotype 5 LZ-20100109 strain) immune group have a geometric mean titer≥1:32 and a protection rate above 80% (4/5); all serotype 5 antibodies of the piglets of the first univalent inactivated vaccine (serotype 1 LC strain) immune group merely have a geometric mean titer of 1:24 and a protection rate of 60% (3/5).

TABLE 2
Protection effects of bivalent inactivated vaccines and univalent inactivated vaccines
of BPS disease against serotype 5

| | | | | | Second | |
|---|---|---|---|---|---|---|
| | | | | First | univalent | |
| | | | | univalent | inactivated | |
| | | | | inactivated | vaccine | |
| | Bivalent | Bivalent | Bivalent | vaccine | (serotype 5 | PBS |
| | inactivated | inactivated | inactivated | (serotype | LZ-20100109 | control |
| Test group | vaccine I | vaccine II | vaccine III | 1 LC strain) | strain) | group |
| Number | 5 | 5 | 5 | 5 | 5 | 5 |
| of piglets | | | | | | |
| Challenge | LZ-20100109 strain | | | | | |
| strain | | | | | | |
| Serotype 5 | 1:42 | 1:42 | 1:37 | 1:24 | 1:42 | |
| antibody | | | | | | |
| titer | | | | | | |
| Number | 0 | 0 | 1 | 2 | 1 | 5 |
| of sick | | | | | | |
| piglets | | | | | | |
| Number | 0 | 0 | 0 | 1 | 0 | 3 |
| of dead | | | | | | |
| piglets | | | | | | |
| Number | 5 | 5 | 4 | 4 | 4 | 0 |
| of healthy | | | | | | |
| piglets | | | | | | |
| Result | 5/5 | 5/5 | 4/5 | 3/5 | 4/5 | 5/5 |
| | Protection | Protection | Protection | Protection | Protection | Incidence |

The above test results show that, in the bivalent inactivated vaccines of the HPS disease, an increased effect exists between the serotype 1 LC strain and the serotype 5 LZ-20100109 strain.

2.4 Cross Protection Test

Seven HPS isolates of serotype 2, serotype 4, serotype 10, serotype 12, serotype 13, serotype 14, and serotype 15 were chosen to prepare challenge bacterial liquids for evaluating cross protection effects of the bivalent inactivated vaccines of the HPS disease against heterologous serotypes and comparing the cross protection effects between the bivalent inactivated vaccines and the univalent inactivated vaccines.

The cross protection test of each EPS isolate included steps of:

randomly choosing 90 healthy susceptible piglets aged between 3-4 weeks, and dividing into 6 groups, 15 piglets each group; respectively intramuscularly injecting 2.0 mL bivalent inactivated vaccine I, II, and III into neck of each piglet of three groups, intramuscularly injecting 2.0 mL first univalent inactivated vaccine (serotype 1 LC strain) into neck of each piglet in one group, intramuscularly injecting 2.0 mL second univalent inactivated vaccine (serotype 5 LZ-20100109 strain) into neck of each piglet in one group, and intramuscularly injecting 2.0 mL PBS into neck of each piglet in one group as a control group; after 3 weeks, injecting the piglets of each group again with the same dose in the same manner for strengthening immunity. After 2 weeks, intraperitoneally injecting an HPS isolate bacterial liquid into the piglets for challenge, wherein a challenge dose of each HPS isolate is showed in Table 3; continuously observing the piglets for 2 weeks and recording the disease incidence and the death of the piglets.

The cross protection effects of the bivalent inactivated vaccines and the univalent inactivated vaccines of the HPS disease against the heterologous serotypes are showed in Table 3. From Table 3, the bivalent inactivated vaccines of the HPS disease has an obviously increased cross protection effect against the serotype 13 of the HPS; and meanwhile, the bivalent inactivated vaccines of the HPS disease has a relatively strong cross protection effect against the serotype 4, the serotype 10, the serotype 12, the serotype 14, and the serotype 15 of the HPS. Thus, the bivalent inactivated vaccines (serotype 1 LC strain+serotype 5 LZ-20100109 strain) of the HPS disease has a wide market application prospect and is worth to be popularized.

TABLE 3
Cross protection effects of bivalent inactivated vaccines and univalent
inactivated vaccines of BPS disease against heterologous serotypes

| | | | | | | Number | |
|---|---|---|---|---|---|---|---|
| | | | Number | Number | Number | of | Cross |
| Challenge | Challenge | | of | of sick | of dead | healthy | protection |
| strain | dose | Test group | piglets | piglets | piglets | piglets | rate |
| Serotype | 7.5 × 10⁹ | Bivalent | 15 | 5 | 0 | 10 | 66.7% |
| 2 | CFU | inactivated | | | | | |
| | | vaccine I | | | | | |
| | | Bivalent | 15 | 5 | 0 | 10 | 66.7% |
| | | inactivated | | | | | |
| | | vaccine II | | | | | |
| | | Bivalent | 15 | 6 | 1 | 9 | 60% |
| | | inactivated | | | | | |
| | | vaccine III | | | | | |
| | | First | 15 | 12 | 2 | 3 | 20% |
| | | univalent | | | | | |
| | | inactivated | | | | | |
| | | vaccine | | | | | |
| | | (serotype 1 | | | | | |
| | | LC strain) | | | | | |
| | | Second | 15 | 7 | 0 | 8 | 53.3% |
| | | univalent | | | | | |
| | | inactivated | | | | | |
| | | vaccine | | | | | |
| | | (serotype 5 | | | | | |
| | | LZ-20100109 | | | | | |
| | | strain) | | | | | |
| | | PBS | 15 | 13 | 3 | 2 | 13.3% |
| | | control | | | | | |
| | | group | | | | | |
| Serotype | 7.5 × 10⁹ | Bivalent | 15 | 5 | 0 | 10 | 66.7% |
| 4 | CFU | inactivated | | | | | |
| | | vaccine I | | | | | |
| | | Bivalent | 15 | 4 | 1 | 11 | 73.3% |
| | | inactivated | | | | | |
| | | vaccine II | | | | | |
| | | Bivalent | 15 | 4 | 0 | 11 | 73.3% |
| | | inactivated | | | | | |
| | | vaccine III | | | | | |
| | | First | 15 | 5 | 0 | 10 | 66.7% |
| | | univalent | | | | | |
| | | inactivated | | | | | |
| | | vaccine | | | | | |
| | | (serotype 1 | | | | | |
| | | LC strain) | | | | | |
| | | Second | 15 | 6 | 1 | 9 | 60% |
| | | univalent | | | | | |
| | | inactivated | | | | | |
| | | vaccine | | | | | |
| | | (serotype 5 | | | | | |
| | | LZ-20100109 | | | | | |
| | | strain) | | | | | |
| | | PBS | 15 | 14 | 4 | 1 | 6.6% |
| | | control | | | | | |
| | | group | | | | | |
| Serotype | 6.0 × 10⁹ | Bivalent | 15 | 5 | 1 | 10 | 66.7% |
| 10 | CFU | inactivated | | | | | |
| | | vaccine I | | | | | |
| | | Bivalent | 15 | 4 | 0 | 11 | 73.3% |
| | | inactivated | | | | | |
| | | vaccine II | | | | | |
| | | Bivalent | 15 | 4 | 0 | 11 | 73.3% |
| | | inactivated | | | | | |
| | | vaccine III | | | | | |
| | | First | 15 | 4 | 0 | 11 | 73.3% |
| | | univalent | | | | | |
| | | inactivated | | | | | |
| | | vaccine | | | | | |
| | | (serotype 1 | | | | | |
| | | LC strain) | | | | | |
| | | Second | 15 | 11 | 3 | 4 | 26.7% |
| | | univalent | | | | | |
| | | inactivated | | | | | |
| | | vaccine | | | | | |
| | | (serotype 5 | | | | | |
| | | LZ-20100109 | | | | | |
| | | strain) | | | | | |
| | | PBS | 15 | 15 | 4 | 0 | 0% |
| | | control | | | | | |
| | | group | | | | | |
| Serotype | 6.0 × 10⁹ | Bivalent | 15 | 3 | 1 | 12 | 80% |
| 12 | CFU | inactivated | | | | | |
| | | vaccine I | | | | | |
| | | Bivalent | 15 | 2 | 0 | 13 | 86.7% |
| | | inactivated | | | | | |
| | | vaccine II | | | | | |
| | | Bivalent | 15 | 4 | 0 | 11 | 73.3% |
| | | inactivated | | | | | |
| | | vaccine III | | | | | |
| | | First | 15 | 5 | 1 | 10 | 66.7% |
| | | univalent | | | | | |
| | | inactivated | | | | | |
| | | vaccine | | | | | |
| | | (serotype 1 | | | | | |
| | | LC strain) | | | | | |
| | | Second | 15 | 2 | 0 | 13 | 86.7% |
| | | univalent | | | | | |
| | | inactivated | | | | | |
| | | vaccine | | | | | |
| | | (serotype 5 | | | | | |
| | | LZ-20100109 | | | | | |
| | | strain) | | | | | |
| | | PBS | 15 | 15 | 9 | 0 | 0% |
| | | control | | | | | |
| | | group | | | | | |
| Serotype | 6.0 × 10⁹ | Bivalent | 15 | 7 | 1 | 8 | 53.3% |
| 13 | CFU | inactivated | | | | | |
| | | vaccine I | | | | | |
| | | Bivalent | 15 | 5 | 0 | 10 | 66.7% |
| | | inactivated | | | | | |
| | | vaccine II | | | | | |
| | | Bivalent | 15 | 6 | 1 | 9 | 60% |
| | | inactivated | | | | | |
| | | vaccine III | | | | | |
| | | First | 15 | 10 | 2 | 5 | 33.3% |
| | | univalent | | | | | |
| | | inactivated | | | | | |
| | | vaccine | | | | | |
| | | (serotype 1 | | | | | |
| | | LC strain) | | | | | |
| | | Second | 15 | 11 | 3 | 4 | 26.7% |
| | | univalent | | | | | |
| | | inactivated | | | | | |
| | | vaccine | | | | | |
| | | (serotype 5 | | | | | |
| | | LZ-20100109 | | | | | |
| | | strain) | | | | | |
| | | PBS | 15 | 13 | 5 | 2 | 13.3% |
| | | control | | | | | |
| | | group | | | | | |
| Serotype | 6.0 × 10⁹ | Bivalent | 15 | 4 | 1 | 11 | 73.3% |
| 14 | CFU | inactivated | | | | | |
| | | vaccine I | | | | | |
| | | Bivalent | 15 | 3 | 0 | 12 | 80% |
| | | inactivated | | | | | |
| | | vaccine II | | | | | |
| | | Bivalent | 15 | 3 | 1 | 12 | 80% |
| | | inactivated | | | | | |
| | | vaccine III | | | | | |
| | | First | 15 | 6 | 1 | 9 | 60% |
| | | univalent | | | | | |
| | | inactivated | | | | | |
| | | vaccine | | | | | |
| | | (serotype 1 | | | | | |
| | | LC strain) | | | | | |
| | | Second | 15 | 6 | 1 | 9 | 60% |
| | | univalent | | | | | |
| | | inactivated | | | | | |
| | | vaccine | | | | | |
| | | (serotype 5 | | | | | |
| | | LZ-20100109 | | | | | |
| | | strain) | | | | | |
| | | PBS | 15 | 10 | 5 | 0 | 0% |
| | | control | | | | | |
| | | group | | | | | |
| Serotype | 7.5 × 10⁹ | Bivalent | 15 | 4 | 0 | 11 | 73.3% |
| 15 | CFU | inactivated | | | | | |
| | | vaccine I | | | | | |
| | | Bivalent | 15 | 4 | 0 | 11 | 73.3% |
| | | inactivated | | | | | |
| | | vaccine II | | | | | |
| | | Bivalent | 15 | 5 | 0 | 10 | 66.7% |
| | | inactivated | | | | | |
| | | vaccine III | | | | | |
| | | First | 15 | 5 | 1 | 10 | 66.7% |
| | | univalent | | | | | |
| | | inactivated | | | | | |
| | | vaccine | | | | | |
| | | (serotype 1 | | | | | |
| | | LC strain) | | | | | |
| | | Second | 15 | 4 | 0 | 11 | 73.3% |
| | | univalent | | | | | |
| | | inactivated | | | | | |
| | | vaccine | | | | | |
| | | (serotype 5 | | | | | |
| | | LZ-20100109 | | | | | |
| | | strain) | | | | | |
| | | PBS | 15 | 15 | 3 | 0 | 0% |
| | | control | | | | | |
| | | group | | | | | |

From Table 3, to the serotype 4, serotype 10, serotype 12, serotype 14, serotype 14, and serotype 15, the univalent inactivated vaccines have a high cross protection rate, and the bivalent inactivated vaccines also have a relatively high cross protection rate; to the serotype 2 and the serotype 13, the univalent inactivated vaccines have a low cross protection rate, while the bivalent inactivated vaccines have a relatively high cross protection rate; especially to the serotype 13, the bivalent inactivated vaccines have a better and increased cross protection effect compared with the univalent inactivated vaccines.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

1-5. (canceled)

6. A method for preparing a bivalent inactivated vaccine of Haemophilus parasuis (HPS) disease, comprising steps of: choosing HPS LC strain and HPS LZ-20100109 strain; activating the HPS LC strain and the HPS LZ-20100109; after activating, respectively inoculating the HPS LC strain and the HPS LZ-20100109 strain into media for culturing; obtaining cultures; inactivating the cultures with formaldehyde solution; and adding aluminum hydroxide gel into the cultures to obtain the bivalent inactivated vaccine of the HPS disease; wherein the HPS LC strain has a deposit number of CGMCC No. 5257 and the HPS LZ-20100109 strain has a deposit number of CGMCC No. 5802.

7. The method for preparing the bivalent inactivated vaccine of the HPS disease, as recited in claim 6, wherein the bivalent inactivated vaccine of the HPS disease is an aluminum hydroxide gel vaccine.

8. The method for preparing the bivalent inactivated vaccine of the HPS disease, as recited in claim 7, wherein the HPS LC strain and the HPS LZ-20100109 strain in the aluminum hydroxide gel vaccine have a total antigen content≥2.5×10⁹ CFU/mL.

9. The method for preparing the bivalent inactivated vaccine of the HPS disease, as recited in claim 7, wherein a ratio of an antigen content of the HPS LC strain to an antigen content of the HPS LZ-20100109 strain in the aluminum hydroxide gel vaccine is (2:3)-(3:2).

10. The method for preparing the bivalent inactivated vaccine of the HPS disease, as recited in claim 8, wherein a ratio of an antigen content of the HPS LC strain to an antigen content of the HPS LZ-20100109 strain in the aluminum hydroxide gel vaccine is (2:3)-(3:2).

11. The method for preparing the bivalent inactivated vaccine of the HPS disease, as recited in claim 7, wherein a ratio of an antigen content of the HPS LC strain to an antigen content of the HPS LZ-20100109 strain in the aluminum hydroxide gel vaccine is 2:3, 1:1, or 3:2.

12. The method for preparing the bivalent inactivated vaccine of the HPS disease, as recited in claim 8, wherein a ratio of an antigen content of the HPS LC strain to an antigen content of the HPS LZ-20100109 strain in the aluminum hydroxide gel vaccine is 2:3, 1:1, or 3:2.
